(19) United States
(12) Patent Application Publication
(10) Pub. No.: US 2008/0091221 A1
(43) Pub. Date: Apr. 17, 2008

(54) Suture management system

(76) Inventors: Linda Brubaker, River Forest, IL (US); Kimberly Kenton, River Forest, IL (US)

Correspondence Address: Loyola University Chicago;Stritch School of Medicine, Office of Research Services, 2160 S. First Ave., Building 120 Rm 400, Maywood, IL, 60153, US

(21) Appl. No.: 11/974,801

(22) Filed: Oct. 16, 2007

Related U.S. Application Data
(60) Provisional application No. 60/851,987, filed on Oct. 16, 2006.

(51) Int. Cl. A61B 17/04

(52) U.S. Cl. 606/148

(57) ABSTRACT

This invention relates to a device used to decrease time spent at surgery by organizing multiple sutures to allow the surgeon to quickly tie knots and which also contains a cutting instrument to cut suture material and a retaining and inventory pad for tracking associated sharps and needles.

### CROSS-REFERENCE TO RELATED APPLICATIONS

This application claims the benefit of the filing date of U.S. Provisional Application No. 60/851,987, filed on Oct. 16, 2006, which is herein incorporated by reference.

### FIELD OF THE INVENTION

This invention relates to a device used to organize multiple sutures during surgery which also contains a cutting instrument to cut suture and a retaining and inventory pad for tracking associated sharps and needles.

### BACKGROUND OF INVENTION

Many types of surgery use multiple interrupted sutures to pull various kinds of tissue away from a surgical site or to close a surgical site. When multiple sutures are used they can become tangled and complicate the surgeon's job and extend the time at surgery, thereby increasing the patients exposure to anesthesia. At the least, tying knots in two ends of a multiple interrupted suture involves extending the time a surgical procedure takes to perform because the two ends of the interrupted suture must be matched up to be properly knotted. Also, when the suturing is performed, the length of suture is cut with the use of scissors by personnel assisting the surgeon, thereby adding time to the surgery as the surgeon presents the suture thread to the assistant to be cut. Finally, the needles and any sharps used during the procedure must be kept track of throughout the surgery to avoid leaving any of them inside the patient.

Various suture organizers have been developed to aid the surgeon with these problems. The Gabbay-Frater organizer is well known for heart surgeries, where the holder holds sutures and their associated surgical needles along with a prosthetic heart valve. Gabbay et al., U.S. Pat. No. 4,185,636, Jan. 29, 1980. Each suture is frictionally retained by a foam insert in a leg, which is permanently attached to a rigid holder. Another device is disclosed in U.S. Pat. No. 3,515,129, to Truhan, where the sutures are held by a device having “wings”. U.S. Pat. No. 5,207,703, to Jain, issued on May 4, 1993, claims a suture organizer having fingerlike projections extending upwards from a plate. The projections loosely hold sutures that are weighted, generally by clamps, keeping them untangled. Another suture organizer disclosed in U.S. Pat. No. 2,692,599 to Creelman, Oct. 26, 1954, is used for hysterectomies, where a spring is mounted on a base taught as the holding means for the sutures. The spring exerts enough force to hold the suture ends in place.

In joint surgeries such as rotator cuff repair surgery, where the patient is neither supine nor motionless, other types of organizers have been taught. Jannot, in U.S. patent application Ser. No. 10/627,364, teaches a holder made of resilient material to hold both sutures and clamps that is useful in a surgical site that may experience motion during surgical procedures. Another suture guide system to prevent tangling of sutures is described in U.S. Pat. No. 6,969,395 B2 to Snyder, issued on Nov. 29, 2005, where an elongated tube is used to hold a pair of sutures while further suturing is made with different suture, which in turn is fixed into another tube. Each pair of suture ends is easily available for knotting, particularly in an arthroscopic shoulder procedure.

Holders of suture material have also been described. Most of these holders are not used as an aid during the surgical procedure itself, but rather for those assisting in surgery to have materials readily at hand. For example, the suture holder of Erickson, described in U.S. Pat. No. 3,819,039, issued Jun. 25, 1974, describes a block of resilient material having a series of parallel slits into which various sutures are positioned to hold them in place until removed for use in the surgery. Another holding device similar to that described by Erickson was claimed in U.S. Pat. No. 5,005,710, issued on Apr. 9, 1991 by Hofer, also consists of a foam block having incisions that are meant to hold various tools.

Another problem seen during surgery is that of keeping track of needles and other surgical sharps once they are used. An accurate count must be made to insure that none of these items are left inside the patient once the surgery is completed. Some systems of keeping this inventory are described in U.S. Pat. No. 4,008,802 issued to Samuel L. Freitag on Feb. 22, 1977, and in U.S. Pat. No. 4,151,913, issued on May 1, 1979, also to Freitag. These are both pads of resilient material that are scored with numbered boxes, making a place where a needle or sharp can be inserted and tallied at a glance. The back of the pad contains an adhesive surface which allows the pad to be affixed to the support table in the surgical suite.

Although these various types of suture retaining means and inventory pads for used needles and surgical sharps are well known, there is a need for a complete suture management system that enables a surgeon to quickly and easily organize untied suture ends together where they are held until being tied into a knot, and which has a means for cutting the suture quickly, and a means to place and keep track of the number of needles and sharps used during surgery, all for the purpose of progressing through a surgical procedure more quickly than is currently possible.

### SUMMARY OF THE INVENTION

It is therefore an object of this invention to provide a suture management system to organize and retain sutures and needles and sharps during surgery, and which is particularly useful for surgeries where the patient is still and supine, such as in urogenital surgery. The inventive suture management system comprises at least one suture holding means designed to hold suture material; a cutting means such as a safety razor held in a holding means to allow the easy cutting of suture material; and a retaining and inventory pad for keeping track of needles and surgical sharps used during surgery. This system is autoclavable, light-weight, inexpensive, and disposable. These and other objects of the invention are achieved by the preferred embodiment disclosed herein.

The first element of the system is a suture holding means comprising at least one square or rectangular block of autoclavable, resilient, flexible foam or sponge material containing a plurality of slits formed in the block. The slits are generally parallel to each other and perpendicular to the bottom wall of the block and are arranged so that the opposing sides of each slit contacts each other with gentle pressure. Each slit will hold one pair of sutures with the gentle pressure so exerted by the opposing sides of each slit. The slits extend through the block top wall, through the body of the foam block, to a depth that is from greater than two times the width of suture material to less than fully through the block and above the bottom wall in order to maintain a cohesive block, as the depth needed is only that which will securely hold one pair of sutures. The resilient, flexible foam material may for example consist of polyurethane foam, PVC foam, polyethylene foam, or foam rubber among other materials. A glue bead or stripe, or a pressure sensitive adhesive strip, may be attached to the bottom wall of the suture holding means and may be covered by a removable paper or plastic covering. Alternatively, a backing sheet may be adhered to the bottom wall of the suture holding means, and the exposed surface of the backing sheet contains a glue bead or stripe, or at least one strip of pressure sensitive adhesive tape, that is covered by a removable paper or plastic covering. Once uncovered, these glue surfaces allow the surgeon to place the suture holding means directly on the drape surrounding the incision site during surgery.

The portions of the foam block (or suture holding means) between the parallel slits, the abutments, are preferably substantially equivalent in size and according to one embodiment of the invention, are rectangular in size. To allow easy access to the slit by the surgeon, a piece of the abutment may be carved on an angle, beginning at the top wall of the abutment near the slit and leading into the slit on a downwardly angle. When this carving is down on each abutment facing a slit, a “V” shape is formed, with the widest part of the “V” being seen on the top wall and the narrowest part of the “V” being the slit found below the top wall in the body of the block. Any shape of carving that would allow easy access to the slit may also be used, such as a half of a square, or half of a circle, with the lowest point leading to the slit. Even a carving where only one opposing abutment is carved so that a low point is below the top wall is acceptable so that the surgeon has a guide to lead to the slit.

In a particularly preferred embodiment, two suture holding means are provided in the suture management system, for use by one surgeon or two surgeons simultaneously suturing opposite sides of an incision site, where one suture holding means is placed between each surgeon and the incision.

The second element of the preferred embodiment of the invention is a retaining and inventory pad for keeping track of needles and surgical sharps used during surgery. A square or rectangular block of resilient foam material, which can be flexible, made from materials as described above for the first element, having a top wall provided with a plurality of needle and sharps receiving zones. Preferably, the zones may be marked with numbers running consecutively from 1 through the total number of zones, although no such markings are necessary to this invention. The zones may be provided as a grid or multiple rows or columns printed or placed on the top wall with ink or a plastic melt. Alternatively, the zones may be carved into the top wall of the foam block. As described above, a removably covered glue bead or stripe, or pressure sensitive adhesive strip may be attached to the bottom wall of the inventory pad, or a backing sheet may be adhered to the bottom wall of the pad, and the exposed surface of the backing sheet contains a glue bead or strip or at least one strip of pressure sensitive adhesive tape that is covered by a removable paper or plastic covering.

The third element of the preferred embodiment of the invention is a cutting means in a holding means. Preferably, the cutting means is a piece of metal or plastic having one surface that is sharp enough to cut cleanly through suture, for example, a safety razor blade having a sharp edge and a dull or “safe” edge, which is held in place by a holding means whereby the blade is held perpendicular to both the bottom wall of the holding means and the draping material around the incision site with the sharp edge facing upward from just above the bottom wall of the holding means and below the top wall of the holding means so that at least a portion of the cutting edge is exposed and available for use by the surgeon to cut the suture. Preferably, the exposed edge is recessed below the top wall of the holding means, so as to prevent the inadvertent cutting of surgical room personnel by coming in accidental contact with the cutting edge. The recession may be made by cutting away enough of the holding means to expose the sharp edge of the cutting means, or by designing the holder to have side walls that both grasp or encase enough of the cutting means to hold it firmly, while having an opening through which the surgeon can use the cutting mean's sharp edge. The sharp edge of the cutting means may also be placed above the top wall of the holding means and fitted with a removable safety cover or with a mechanism to withdraw the blade into the holding means when not in use. The holding means may be molded plastic into which the metal or plastic sharp edge or blade is permanently contained, such as seen with disposable shaving razors. Alternatively, the holding means may be the same material as is the block of resilient, flexible foam as described above. A cover that will flip open or is removable will optionally cover at least the exposed blade. The base or bottom wall of the holding means must have a surface area great enough to allow the cutting surface to remain upright when standing alone or attached to a drape. As described above in the first and second elements, a removably covered glue bead or stripe or pressure sensitive adhesive tape may be affixed to the bottom wall of the holding means or to the outside surface of a backing material used on the bottom wall of the holding means. In a particularly preferred embodiment, the holding means is a rectangular block of resilient foam material of a dimension large enough to encase a safety razor blade, encasing the safety razor blade, while exposing a section of the cutting edge through a notch cut into the foam block, and providing a removable cover over the top of the holding means. One strip of removably covered adhesive is placed on the bottom wall surface of the holding means, and another is placed on the outside side wall perpendicular to the bottom wall surface. The holding means is attached to a side wall of the retaining and inventory pad, and when in use at the surgical site, the bottom adhesive strip is uncovered and placed on the surgical drape. In this manner, the holding means is removably attached to the retaining and inventory pad to insure stability of the cutting surface of the razor blade, while also being attached to the drape. Adhesive tape can be attached to a holding means made of any suitable material, such as plastic or foam.

An optional fourth element of the suture management device is a thin sheet of plastic or paper on which the first three elements are placed and adhered to with glue or pressure sensitive adhesive tape, to allow for easy packaging of the suture management system.

### BRIEF DESCRIPTION OF THE DRAWINGS

FIG. 1 is a top down view of the suture management system components of one retaining and inventory pad, one holding means with a cutting means, in this case a safety razor blade, and a cover, and two suture holding means, all placed around a piece of material.

FIG. 2 is a view showing FIG. 1 from a side angle, with each component arranged on a drape surrounding material to be sutured.

FIG. 3 shows the use of the suture management system in place during a surgical procedure.

FIG. 4 shows a side view of a suture holding means where the top wall is cut on an angle.

### DETAILED DESCRIPTION OF THE PREFERRED EMBODIMENT

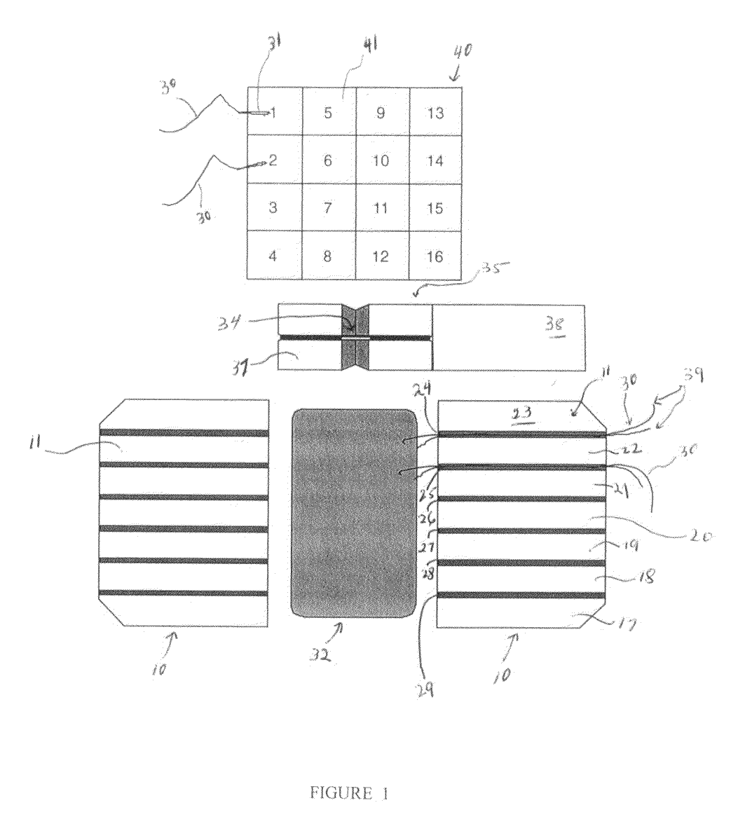

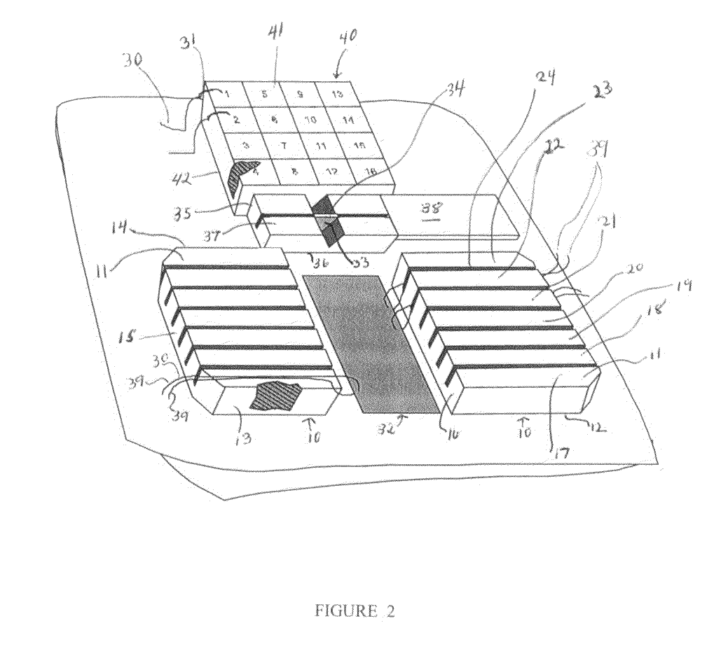

Referring now to FIG. 1 and FIG. 2, shown are two of the first element of the suture management system, each suture holding means 10 (also called block 10) is made from a generally rectangular shaped block of autoclavable, resilient, flexible material. The block has a top wall 11, bottom wall 12, end walls 13 and 14, and outer side wall 15 and inner side wall 16. Although in the preferred embodiment the suture holding means is rectangular, any shape that performs as described herein, such as a square or triangular shape, is acceptable. Also, as seen in FIG. 4, the top wall 11 of the suture holding means 10 may be shaped to be angular instead of parallel to the bottom wall, and the angle having an apex where top wall 11 meets outer side wall 15, which is higher than inner side wall 16. Another embodiment envisioned is where top wall 11 meets inner side wall 16 and inner side wall 16 is higher that outer side wall 15. And still another embodiment is where top wall 11 is made to the shape of an upside down “V”, having a apex near the center of top wall 11, extending from end wall 13 to end wall 14.

The resilient, flexible material of suture holding means 10 must be able to withstand temperatures reached during conventional autoclaving to sterilize it. By way of example, the material may be sponge rubber, polyurethane, or polyethylene foam.

A row of side-by-side abutments 17-23 are found in the area between a series of parallel slits 24-29. Although in FIG. 1 and FIG. 2 is shown seven abutments and 6 slits, the number of abutments and slits may vary depending on different requirements, for example, of the number of sutures to be managed. The slits 24-29 may be simple slits, going through the suture holding means 10 as far as desired, as long as they do not cut through the bottom wall 12, so that the integrity of the suture holding means 10 is maintained. The slits 24-29 may be indicated by an ink or other marking at the point where the slit cuts through the top wall 11, or by an indicator notch or groove that may be made in the top wall 11 abutments that lead into the rest of the slit, thereby making identification of the placement of each slit easy for the surgeon. The adjacent faces of the side-by-side abutments generally contact each other, while exerting light pressure on each other. One time that the adjacent faces of the side-by-side abutments will not contact each other is when suture material is placed in the slit, and the pressure of the faces will be applied to the suture material. Another occurs in the area found at the top wall 11 when a notch or groove is made in either or both abutments surrounding a slit and the sides of the slit are cut away to form the notch or groove.

During a surgical procedure, suture material 30 is threaded through a needle 31 and the needle with this suture material 30 is passed through the tissue to be sewn, or sutured, together. On occasion, tissue supporting material 32, such as a woven fabric, is also incorporated into the tissue to be sutured. Once the needle and attached suture material is passed through all layers of tissue and possibly tissue supporting material, the suture must be cut to free the needle 31 from the site, while leaving enough suture material available to tie a knot to close the suture.

The third element of the preferred embodiment is a cutting means in a holding means. Preferably, a safety razor blade-type piece of metal 33, having at least one sharp edge 34 which can easily cut through suture material 30 is contained in a holding means 35. This holding means 35 holds the blade in a position whereas at least a portion of the sharp edge 34 is available to the surgeon to cut the suture material. In this preferred embodiment, the razor blade 33 is held perpendicular to the bottom wall 36 of the holding means 35, and the sharp edge 34 is facing upward towards the surgeon, who when bringing the suture material into contact with the blade, cuts it. Preferably, the holding means 35 maintains the razor blade 33 at a distance below the top wall 37 of the holding means 35, in order to protect people working with the suture management system from accidentally being cut. Additionally, a cover 38 may be provided that is in place over the top wall 37 of the holding means and in particular over the razor blade 33 and that may be left in place prior to use at the suturing site. The holding means 35 and cover 38 may be made of the same flexible, resilient material as is the suture holding means 10, or may be made of any moldable plastic or other material that can withstanding autoclaving temperatures. A substantial benefit is seen in time savings during the surgery just by eliminating the need of the surgeon to hold out the suture materials and having a second party cut it.

Once the suture material is ready to be cut, the surgeon can easily and quickly press it against the exposed sharp edge 34 of cutting means 33 in holder 35, thereby separating the needle 31 and extra suture material from the suture made in the patient. The needle 31 is placed into the second element of the preferred embodiment, the retaining and inventory pad 40 described below. The length of suture material attached to the suture site has two cut ends, which are then placed into any of available slits 24-29 of the suture holding means 10. Preferably, the first slit 24 is used for the first suture to be knotted in a row, the second slit 25 used for the second suture to be knotted in a row, etc., until each set of suture material is placed and held in a slit in the suture holding means 10. The length of suture material left to be knotted must be longer than the length of the slit, so that the free ends 39 of the suture material are easily available to the surgeon after being placed into the holding means 10.

If the top wall 11 of the suture holding means 10 has been made on an angle wherein for example outer side wall 15 reaches a height greater than inner side wall 16, and the apex of the angle is at the junction of outer side wall 15 and top wall 11, thereby in essence forming a point, such as seen in FIG. 4, the surgeon will need to only locate the beginning of the slit 29 (for example) at the apex into which to begin pressing down on the suture material pair to introduce the suture material pair into the full length of slit 29. In the same manner, suture holding means 10 can be formed to have its top wall I 1 in the shape of an upside down “V”, with its apex roughly centered in the top wall 11 and outer side wall 15 and inner side wall 16 being approximately equal to each other and being shorter that the center of the apex of top wall 11. This too would allow the surgeon to quickly locate an insertion point for the suture material and merely press down at the apex to insert the suture material in slit 29.

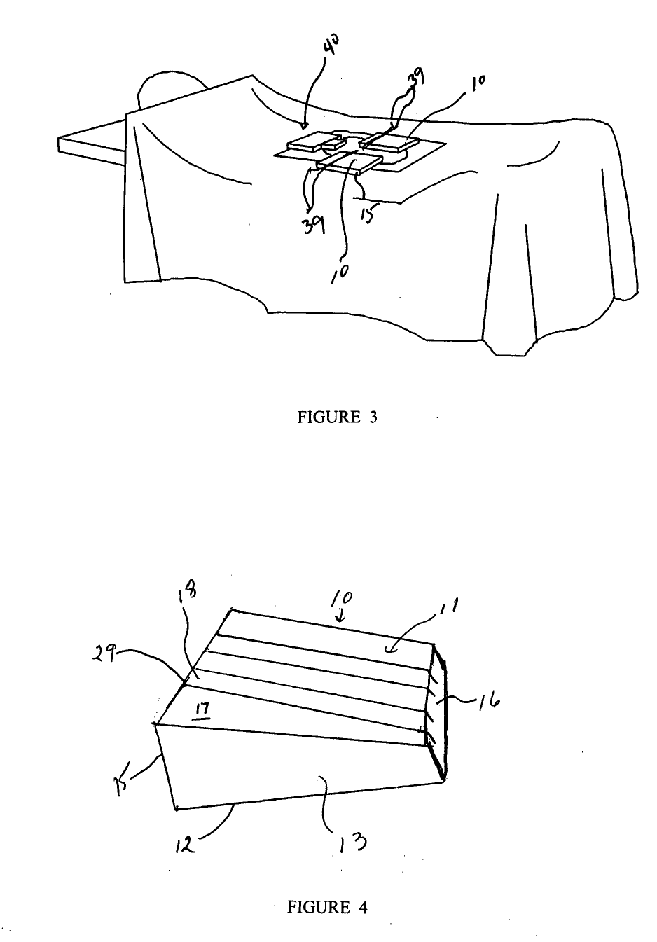

In FIG. 3, there is shown a representation of a patient undergoing surgery, with the inventive suture management system being used. As can be seen, one length of cut suture material having two free ends 39, which has been used to sew together tissue with a portion remaining in the patient, has been placed in a slit in one of a suture holding means 10 on one side of the area being sutured, with a second length of cut suture material also having two free ends 39, also used to sew up tissue and having a portion still in place inside the patient, has been placed in a slit in another suture holding means 10 on the other side of the area being sutured, and in both cases free ends 39 are seen extending past the outer side walls 15. This is also seen in FIG. 1 and FIG. 2 where the suture material is seen in place in slit 24. In this case, it is envisioned that one surgeon is working on one side of the patient and another on the other side. By being able to manage their own sutures, the two surgeons have a clear, tangle-free area of sutures ready to be knotted. Generally, once all sutures have been made that are desired to be made and the suture material cut and its paired ends placed in a slit of the suture holding means, the surgeon or other responsible party can quickly and easily lift one pair of ends 39 from the slit it is being held in, tie the knots, and go on to the next suture. The time that is saved by not having to untangle each set of ends and find the matching set, by not having to identify the matching set of ends by attaching them together with an instrument such as a clamp or any other type of attaching means, can be substantial, particularly in surgeries where a substantial number of sutures are needed during the procedure.

The second element of the present invention is a retaining and inventory pad, or pad 40. Pad 40 may be of any shape, but preferably a rectangular or square shaped block of resilient, flexible material, such as that described for the suture holding means 10, and that is susceptible to being autoclaved. It may also be any material that can be nonflexible, but that can easily be punctured, such as polystyrene, as long it can withstand autoclave conditions to insure sterility. It consists of a top wall 41, a bottom wall 42, and as many side walls as needed for the shape chosen. A circular shape will have one side wall, a triangular shape will have three walls, and rectangles and squares will have four side walls. The top wall 41 contains markings that are individual cells, such as those found in a grid, or individual columns or rows. Each cell, column, or row is known as a zone. The marking of zones may be made by any suitable printing ink or a plastic melt, or may be made by molding the resilient, flexible material with ridges or channels, thereby delineating individual zones. Each zone is consecutively numbered, as shown on the top wall 41 of FIG. 1 and FIG. 2. When a needle 31 or any sharp used during surgery is no longer needed, it is placed into a single zone, in a consecutive manner, thereby allowing the surgeon or other operating room personnel to keep track of the number of needles and sharps taken from the patient and to insure that none of them have been left inside the patient.

Desirably, each element of the inventive suture management system, the suture holding means 10, the retaining and inventory pad 40, and the holding means 35 is provided with at least one, but preferably two or more, pressure sensitive adhesive strips which may be attached to the bottom walls 12, 42, and 36, covered by a removable paper or plastic covering. A bead or strip of glue that is also removably covered are examples of other adhesive applications which may also be used. When the covering is removed, the element may be affixed to the draping surrounding the incision site. Alternatively, the bottom wall of any element may be attached by adhesive to a relatively stiff but still flexible backing sheet, such as paperboard or a thin plastic sheet, to give more body to that element. The exposed surface of the backing sheet will have at least one strip of pressure sensitive adhesive tape secured to it, covered by a removable paper or plastic covering, which when removed allows the element to be affixed to the draping surrounding the incision site.

Additionally, all three elements of the suture management system may be mounted on a paperboard or plastic base sheet for ease in packaging as a unit. In this case, a removably covered adhesive in the form of glue or tape would be placed on each individual element either on the bottom wall or the backing sheet so that each element would be affixed to the base sheet and then could be inserted into packaging that allows the whole system and package to be sterilized.

We claim:

1. A suture management system comprising:
a) at least one suture holding means designed to hold suture material;
b) a cutting means held in a holding means to allow the easy cutting of suture material; and
c) a retaining and inventory pad for keeping track of needles and surgical sharps used during surgery,
wherein each of a, b, and c above have an adhesive attached to a bottom wall of said suture holding means, said holding means, and said retaining and inventory pad.

2. A suture management system according to claim 1, wherein said suture holding means, said holding means, and said retaining and inventory pad are each comprised of a block of autoclavable, resilient, flexible foam or sponge material.

3. A suture management system according to claim 2, wherein said adhesive is a glue bead or stripe, or an adhesive strip, covered with a removable piece of plastic or paper.

4. A suture management system according to claim 2, wherein said suture holding means comprises:
a) said block having a top wall, a bottom wall, opposing end walls and an outer side wall and an inner side wall;
b) plurality of parallel slits cut into said block thereby forming plural abutments whose opposing faces generally contact each other on either side of said slits, wherein said slits pass entirely through said top wall and into the body of said block to a depth of from at least the thickness of two pieces of suture material to no further than immediately above said bottom wall, in order to maintain the integrity of said block; and
c) wherein said slits maintain a pressure on suture material introduced into said slits, holding the suture material in place through the contacting opposing faces of said abutments.

5. A suture management system according to claim 2, further comprising:
a) said cutting means comprising a piece of metal or plastic having a sharp edge capable of cutting suture material which is held in place by a holding means whereby said sharp edge of said piece of metal or plastic is held perpendicular to the bottom wall of the holding means with the sharp edge facing upward, providing at least a portion of the cutting edge exposed to the surgeon;
b) the bottom wall of the holding means provides stability to maintain said cutting means upright; and
c) the holding means preferably holds said cutting means in a manner so that said sharp edge is below the top wall of said holding means, while allowing access to said sharp edge; and optionally,
d) a removable cover over said sharp edge.

6. A suture management system according to claim 2, further comprising said retaining and inventory pad comprising said block in a square or rectangular shape, having a top wall provided with zones as a grid, columns, or rows, and optionally, said zones marked with numbers running consecutively from 1 through the total number of zones, printed or placed on the top wall with ink or a plastic melt.

7. A suture management system according to claim 6, further comprising a thin sheet of plastic or paper on which the first three elements are placed and adhered to with glue or pressure sensitive adhesive tape, to allow for easy packaging of the suture management system.
